(19) United States
(12) Patent Application Publication
(10) Pub. No.: US 2025/0082180 A1
(43) Pub. Date: Mar. 13, 2025

(54) ENDOSCOPE

(71) Applicant: OLYMPUS MEDICAL SYSTEMS CORP., Tokyo (JP)

(72) Inventor: Tomonori AWAJIKAWA, Tokyo (JP)

(73) Assignee: OLYMPUS MEDICAL SYSTEMS CORP., Tokyo (JP)

(21) Appl. No.: 18/828,207

(22) Filed: Sep. 9, 2024

Related U.S. Application Data
(60) Provisional application No. 63/537,600, filed on Sep. 11, 2023.

(51) Int. Cl. A61B 1/00; A61B 1/12

(52) U.S. Cl. CPC A61B 1/00091; A61B 1/126

(57) ABSTRACT

An endoscope includes: an observation window disposed in a distal end portion of an insertion portion; a nozzle disposed in the distal end portion; and a cover configured to cover the distal end portion, a distal end surface of the cover includes: a first surface surrounding the observation window, the first surface having a first planar shape when viewed in a cross-section taken about a line connecting the observation window and the nozzle; a second surface disposed radially outside the first surface, the second surface having a curved shape when viewed in the cross-section; and a third surface disposed radially outside of the second surface, the third surface having a second planar shape.

### RELATED APPLICATION DATA

This application is based on and claims priority under 35 U.S.C. § 119 to U.S. Provisional Application No. 63/537,600, filed on Sep. 11, 2023, the entire contents of which are incorporated herein by reference.

### BACKGROUND

### 1. Field of the Disclosure

The present disclosure relates to an endoscope including a distal end cover in a distal end portion of an insertion portion.

### 2. Description of the Related Art

Japanese Patent Application Laid-Open Publication No. 2003-210388 discloses an endoscope in which a distal end cover provided on a distal end surface of an insertion portion includes an annular inclined portion surrounding an observation window and fluid is ejected toward the inclined portion from an air/water feeding nozzle.

### SUMMARY

An endoscope of an embodiment of the present disclosure includes: an observation window disposed in a distal end portion of an insertion portion; a nozzle disposed in the distal end portion; and a cover that covers the distal end portion, in which a distal end surface of the cover includes: a first surface surrounding the observation window, the first surface having a first planar shape when viewed in a cross-section taken about a line connecting the observation window and the nozzle; a second surface disposed radially outside the first surface, the second surface having a curved shape when viewed in the cross-section; and a third surface disposed radially outside of the second surface, the third surface having a second planar shape.

### BRIEF DESCRIPTION OF THE DRAWINGS

FIG. 1 is a perspective view of an endoscope system including an endoscope of an embodiment;

FIG. 2 is a plan view of a distal end surface of an endoscope of a first embodiment;

FIG. 3 is a cross-sectional view taken along line III-III of FIG. 2;

FIG. 4 is a partial enlarged view of a surface shape of a distal end cover of the endoscope of the first embodiment;

FIG. 5 is a plan view of the distal end surface of an endoscope of a modification 1 of the first embodiment;

FIG. 6 is a partial plan view of the distal end surface of the endoscope of the modification 1 of the first embodiment;

FIG. 7 is a view showing a change in a width of a distal end cover of the endoscope of the modification 1 of the first embodiment;

FIG. 8 is a partial plan view of the distal end surface of an endoscope of a modification 2 of the first embodiment;

FIG. 9 is a partial plan view of the distal end surface of an endoscope of a modification 3 of the first embodiment;

FIG. 10 is a partial plan view of the distal end surface of an endoscope of a modification 4 of the first embodiment;

FIG. 11 is a partial plan view of the distal end surface of an endoscope of a modification 5 of the first embodiment;

FIG. 12 is a partial plan view of the distal end surface of an endoscope of a modification 6 of the first embodiment;

FIG. 13 is a partial plan view of the distal end surface of an endoscope of a second embodiment;

FIG. 14 is a partial plan view of the distal end surface of an endoscope of a third embodiment; and

FIG. 15 is a partial plan view of the distal end surface of an endoscope of a modification of the third embodiment.

### DETAILED DESCRIPTION

Hereinafter, endoscopes of embodiments of the present disclosure will be described using the drawings. Note that in the following description, the drawings based on the embodiments are schematic illustrations. In the drawings, the relation between the thickness and the width of each portion, the ratio in thickness of each portion, and the like differ from the actual relation, ratio, and the like. There are also some portions with different dimensional relations and ratios among the drawings. Illustration of and assignment of reference signs to some of the components will be omitted in the drawings.

### <Endoscope System>

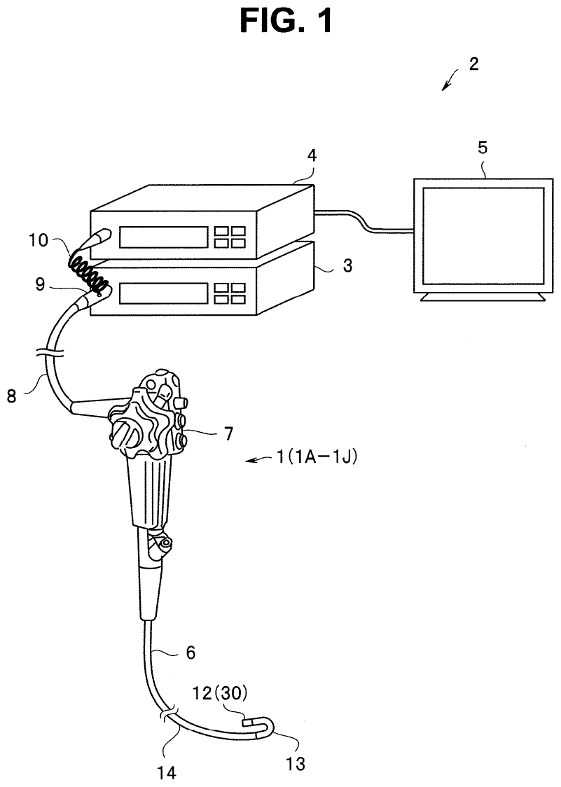

As shown in FIG. 1, an endoscope system 2 includes an endoscope 1, a light source device 3, a video processor 4, and a monitor 5 of an embodiment.

The endoscope 1 includes an insertion portion 6 to be inserted into a body cavity, an operation portion 7, and a universal cord 8. The insertion portion 6 is continuously provided with a distal end portion 12, a bending portion 13, and a flexible portion 14 in this order from the distal end side. The universal cord 8 is provided with a connector 9 connected to the light source device 3, and an electric cable 10 connected to the video processor 4 extends from the connector 9. An image pickup signal of an image picked up by the endoscope 1 is subjected to image processing by the video processor 4 and an endoscope image is displayed on the monitor 5.

### First Embodiment

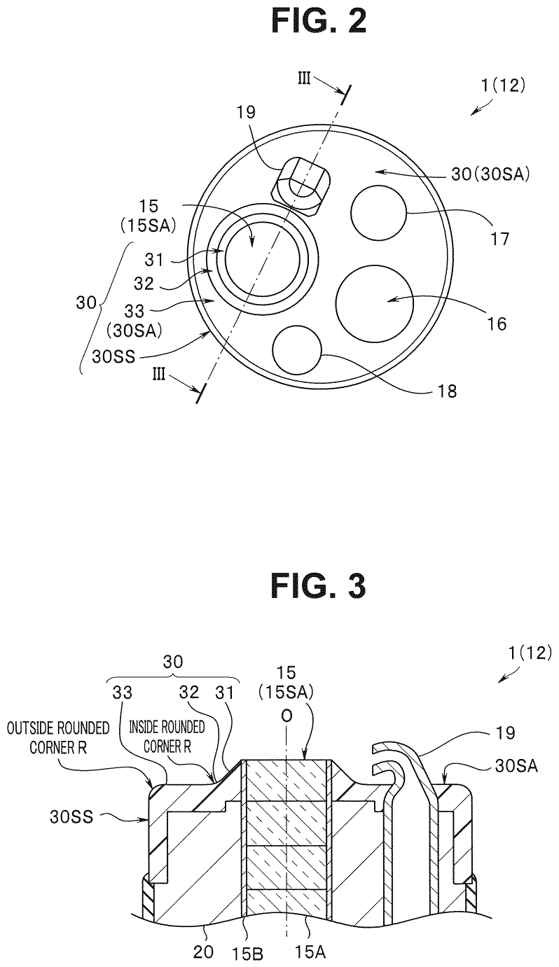

FIG. 2 is a front view of the distal end portion 12 of the endoscope 1 of the embodiment as viewed from a side of a distal end surface. An observation window 15 including an objective lens, a channel opening 16, illumination windows 17, 18, and an air/water feeding nozzle 19 are disposed on the distal end surface of the distal end portion 12. The distal end portion 12 is covered by a distal end cover 30 including a plurality of openings respectively corresponding to the observation window 15, the channel opening 16, and the illumination windows 17, 18.

The observation window 15 is a forefront surface of an image pickup optical system 15A including a lens holding member 15B. An adhesive (not shown) may be disposed between the image pickup optical system 15A and the lens holding member 15B. In the drawings below, an outer surface 15SA of the observation window 15 indicates the forefront surface including the image pickup optical system 15A, the adhesive, and the lens holding member 15B. In the air/water feeding nozzle 19, an opening C19 that ejects fluid in a direction toward the observation window 15 is disposed.

A distal end surface 30SA of the distal end cover 30 includes an annular first area 31 (first surface) surrounding the observation window 15, an annular second area 32 (second surface) in contact with the first area 31 on the inner circumference, and a third area 33 (third surface) in contact with an outer circumference of the second area 32. The second area 32 is formed on an outer side of the first area 31 and the third area 33 is formed on an outer side of the second area 32.

The third area 33 is a plane parallel to the outer surface 15SA of the observation window 15. The outer surface 15SA of the observation window 15 projects by, for example, 0.3 mm relative to the third area 33. In other words, there is a step between the third area 33 and the outer surface 15SA of the observation window 15. Therefore, the distal end cover 30 includes an inclined surface between the third area 33 and the outer surface 15SA of the observation window 15. The inclined surface is composed of the first area 31 as a first inclined surface (first planar shape) formed of a plane and the second area 32 as a second inclined surface formed of a curved surface.

FIG. 3 is a cross-sectional view taken along line III-III of FIG. 2, showing a cross-section from the air/water feeding nozzle 19 to the observation window 15. As shown in FIG. 3, the distal end portion 12 is provided with a distal end rigid member 20 on which the image pickup optical system 15A and the like are disposed and the distal end cover 30 covers the top of the distal end rigid member 20.

An inner circumference of the first area 31 (outer circumference of the observation window 15), an outer circumference of the first area 31 (inner circumference of the second area 32), and an inner circumference of the third area 33 are all circles concentric about a center C15 of the observation window 15.

The distal end cover 30 covers a part of an outer circumference surface of the distal end rigid member 20 as well as the distal end surface of the distal end rigid member 20. A boundary area between the distal end surface 30SA and an outer circumference surface 30SS of the distal end cover 30 is a so-called outside rounded corner area chamfered with a curved surface.

Water ejected from the opening of the air/water feeding nozzle 19 in the direction toward the observation window 15 bumps against the first area 31 to be widely spread and flows over the entire outer surface 15SA of the observation window 15. The water flowing over the outer surface 15SA of the observation window 15 converges, halfway in flowing down the first area 31, to the extent that the width is at the same level as the width of the opening of the air/water feeding nozzle 19.

However, there is a region where due to the step between the outer surface 15SA of the observation window 15 and the third area 33, it is difficult for the water ejected from the air/water feeding nozzle 19 to flow in the direction toward the observation window 15 along the inclined surface (second area 32).

For example, after water feeding, the water is likely to remain in a lower portion of the step near the air/water feeding nozzle 19. When air is fed to remove water droplets adhering to the outer surface 15SA of the observation window 15, the water remaining in the lower portion of the step is blown up by the fed air, thereby adhering to the observation window 15. In other words, despite the air feeding to remove the water droplets adhering to the outer surface 15SA of the observation window 15, the water is resupplied to the outer surface 15SA, which results in inefficient air feeding.

Further, when the water flowing over the outer surface 15SA of the observation window 15 flows down to the third area 33 via the first area 31 as the inclined surface, the water droplets are likely to gather in the lower portion of the step. In an endoscope including an optical system with a wide angle of view, the water droplets could occasionally be seen on an image.

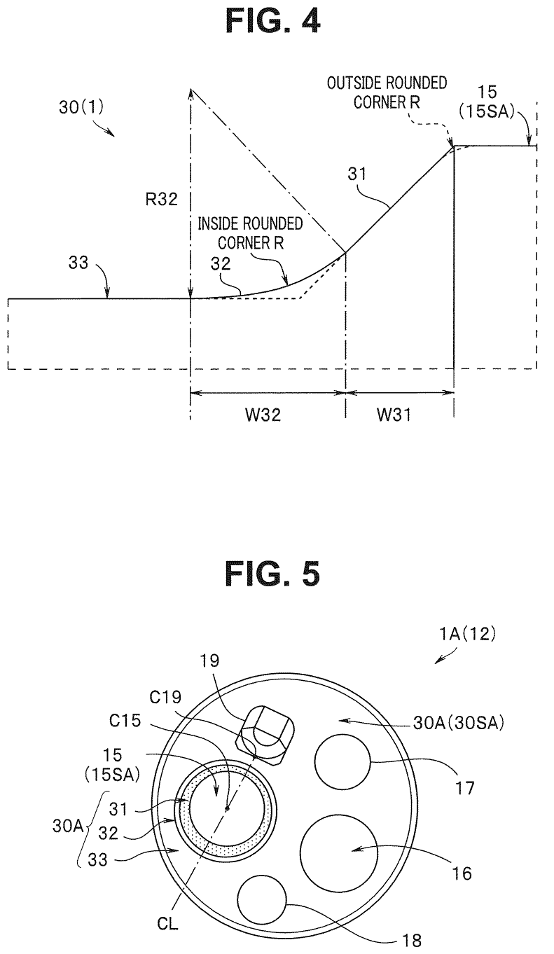

As shown in FIG. 4, the distal end cover 30 of the endoscope 1 includes the second area 32 as an inside rounded corner area at a boundary between the first area 31 and the third area 33. Therefore, water is unlikely to remain at the boundary between the first area 31 and the third area 33. Accordingly, the air/water feeding of the endoscope 1 is efficient.

A width W31 of the first area 31 and a width W32 of the second area 32 are, for example, around 1 mm. For example, the width W31 of the first area 31 is a length of the first area 31 in a direction along the shortest line connecting the center C15 of the observation window 15 and the first area 31.

Since the distal end cover 30 includes the second area 32 formed of a curved surface with a radius of curvature R32 of more than 0.5 mm, generation of the remaining water is prevented. Note that in order to prevent the outer diameter of the distal end portion 12 of the endoscope 1 from being enlarged, the radius of curvature R32 of the second area 32 is preferably less than 2 mm.

Note that the inside rounded corner area is occasionally formed between the first area 31 that is a plane as an inclined surface and the third area 33 that is a plane as a flat surface (second planar shape). The inside rounded corner area has the small radius of curvature R32, thereby having a small effect of prevention of remaining water generation, but is included in the second area.

Note that as denoted by a broken line in FIG. 4, the outside rounded corner area may be formed between the first area 31 and the outer surface 15SA of the observation window 15.

### Modifications of First Embodiment

Endoscopes 1A to 1F of modifications of the first embodiment are similar to and have the same effects as the effects of the endoscope 1. Thus, in the following description, the components having the same functions as the functions of the components of the endoscope 1 are assigned the same reference signs and the descriptions will be omitted.

### Modification 1 of First Embodiment

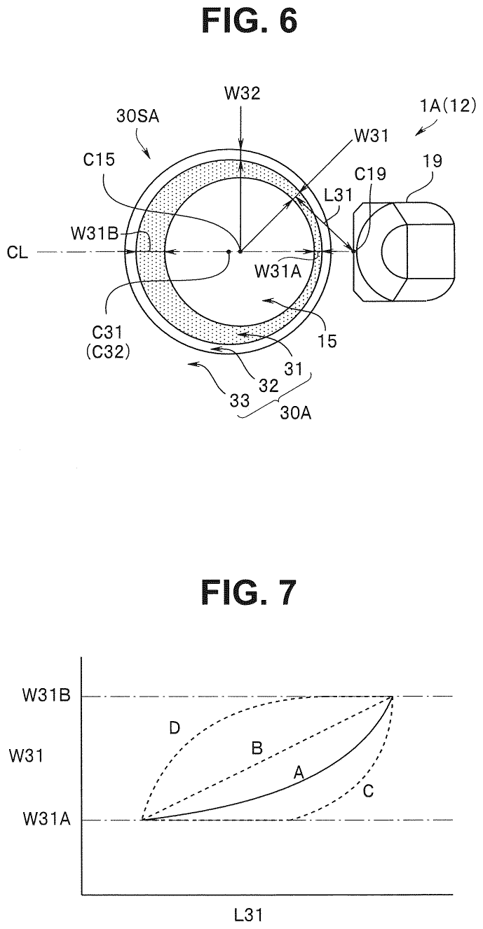

In the distal end cover 30 of the endoscope 1, the width W31 of the annular first area 31 is substantially the same throughout the entire circumference. While on the other hand, as shown in FIG. 5 and FIG. 6, in the distal end cover 30 of the endoscope 1A of the present modification, a width W31B (first width) of the first area 31 where a distance L31 from the air/water feeding nozzle 19 to an outer periphery of the first area 31 is longest is greater than a width W31A (second width) of the first area 31 where the distance from the air/water feeding nozzle 19 to the outer periphery of the first area 31 is shortest.

Note that as shown in FIG. 6, for example, the width W31 of the first area 31 is the length of the first area 31 in the direction along the shortest line connecting the center C15 of the observation window 15 and the first area 31. The distance L31 is a length between the center of the width W31 in the direction along the shortest line and the center of the opening C19 of the air/water feeding nozzle 19. Hereinafter, the “center of the opening C19” is referred to as the “opening C19.”

Note that in a distal end cover 30A, the width W31 of the first area 31 gradually increases as the distance L31 from the air/water feeding nozzle 19 becomes longer (FIG. 7, A).

The outer circumference (inner circumference of the first area 31) of the observation window 15, the outer circumference (inner circumference of the second area 32) of the first area 31, and the outer circumference (inner circumference of the third area 33) of the second area 32 are all circular. A center C31 of the first area 31 and a center C32 of the second area 32 are at the same position on a center line CL, but are positioned farther from the air/water feeding nozzle 19 as compared to the center C15 of the observation window 15.

In the endoscope 1A, the water flowing over the outer surface 15SA of the observation window 15 is more unlikely to remain in the lower portion of the step as compared to the endoscope 1. Therefore, even in the endoscope having the optical system with a wide angle of view, water droplets are not seen on an image.

Note that as shown in FIG. 7, in the endoscope 1A, the width W31 of the first area 31 gradually increases in a curve as the distance L31 from the air/water feeding nozzle 19 becomes longer (A). While on the other hand, the width W31 may linearly increase relative to the distance L31 (B). Further, at least a portion of the width W31 may increase relative to the distance L31 (C, D).

For the efficient air/water feeding, the width W31B is preferably more than 1.2 times the width W31A.

### Modification 2 of First Embodiment

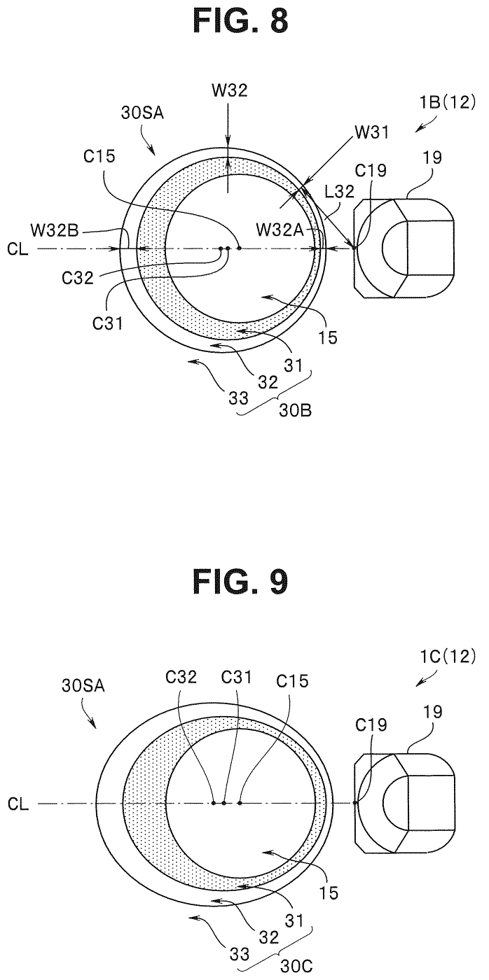

As shown in FIG. 8, in a distal end cover 30B of the endoscope 1B of the present modification, the width W32 of the second area 32 also increases as a distance L32 from the air/water feeding nozzle 19 becomes longer. Therefore, a width W32B (third width) of the second area 32 where the distance L32 from the air/water feeding nozzle 19 to an outer periphery of the second area 32 is longest is greater than a width W32A (fourth width) of the second area 32 where the distance from the air/water feeding nozzle 19 to the outer periphery of the second area 32 is shortest. For the efficient air/water feeding, the width W32B is preferably more than three times, particularly, more than five times the width W32A.

The outer circumference of the first area 31 and the outer circumference of the second area 32 are both circular. The center C32 of the second area 32 is positioned farther from the opening C19 of the air/water feeding nozzle 19 as compared to the center C31 of the first area 31 on the center line CL. Conversely, the center C15 of the observation window 15, the center C31 of the first area 31, and the center C32 of the second area 32 are positioned on the same line (center line CL) and become closer to the opening C19 of the air/water feeding nozzle 19 in the aforementioned order.

In the endoscope 1B, the water flowing over the outer surface 15SA of the observation window 15 is more unlikely to remain in the lower portion of the step as compared to the endoscope 1A, when flowing down the second area 32 to the third area 33. Therefore, even in the endoscope having the optical system with a wide angle of view, water droplets are not seen on an image.

### Modification 3 of First Embodiment

In a distal end cover 30C of the endoscope 1C of the present modification shown in FIG. 9, the outer circumference (inner circumference of the second area 32) of the first area 31 and the outer circumference of the second area 32 are elliptic. The center C31 of the first area 31 and the center C32 of the second area 32 are positioned on the center line CL. Note that the center of the ellipse means a midpoint between two focal points. The center C32 has a longer distance from the air/water feeding nozzle 19 than the center C31.

In the distal end cover 30C, the width W31 of the first area 31 more significantly changes as compared to the distal end cover 30B depending on the distance from the air/water feeding nozzle 19. Thus, in the endoscope 1C, the water flowing over the outer surface 15SA of the observation window 15 is more unlikely to remain in the lower portion of the step as compared to the endoscope 1B. Therefore, even in the endoscope having the optical system with a wide angle of view, water droplets are not seen on an image.

Note that the shape of the first area 31 and the shape of the second area 32 may be substantially circular or substantially elliptic, instead of being in a perfect circle or an ellipse.

### Modification 4 of First Embodiment

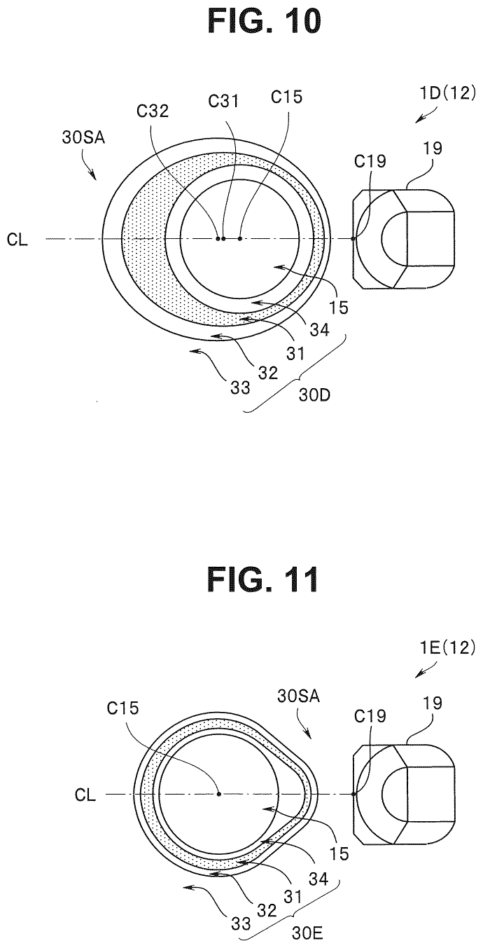

The distal end surface 30SA of a distal end cover 30D of the endoscope 1D of the present modification shown in FIG. 10 includes an annular fourth area 34 (fourth surface) between the observation window 15 and the first area 31. The outer circumference of the observation window 15 and an inner circumference of the fourth area 34 contact with each other and the inner circumference of the first area 31 and an outer circumference of the fourth area 34 contact with each other. The fourth area 34 is a plane parallel to the outer surface 15SA of the observation window 15 and the third area 33. The outside rounded corner area may be formed at a boundary between the first area 31 and the fourth area 34. In the modifications 1 and 2 also, the fourth area 34 may be formed.

In the fourth area 34 of the distal end cover 30D shown in FIG. 10, a width W34 is constant, but in the fourth area 34, the width W34 may vary.

### Modification 5 of First Embodiment

In the distal end surface 30SA of a distal end cover 30E of the endoscope 1E of the present modification shown in FIG. 11, the fourth area 34 projects toward the air/water feeding nozzle 19. Accordingly, a portion of the first area 31 and a portion of the second area 32 also project toward the air/water feeding nozzle 19.

In the endoscope 1E, the flow rates of the water and the air ejected from the air/water feeding nozzle 19 flowing toward the observation window 15 increase as compared to the endoscope 1 and the like. Accordingly, the endoscope 1E has a further higher cleaning efficiency than the endoscope 1 and the like.

### Modification 6 of First Embodiment

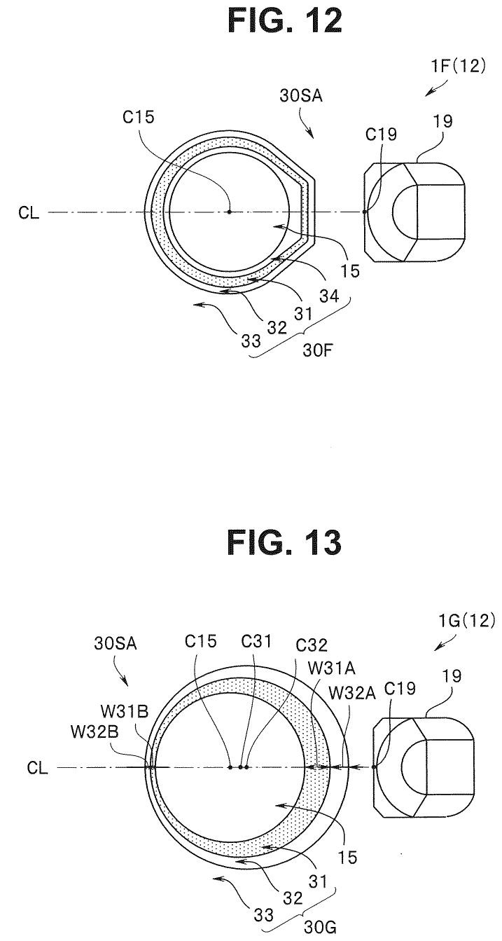

In the distal end surface 30SA of a distal end cover 30F of the endoscope 1F of the present modification shown in FIG. 12, the fourth area 34 projects toward the air/water feeding nozzle 19 and an outer edge facing the air/water feeding nozzle 19 is a straight line. Accordingly, the outer edges facing the air/water feeding nozzle 19 of the first area 31 and the second area 32 are also straight lines. In other words, regions facing the air/water feeding nozzle 19 of the first area 31 and the second area 32 are substantially rectangular. The portion of the first area 31 and the portion of the second area 32 are each equidistant from the air/water feeding nozzle 19.

In the endoscope 1F, the flow rates of the water and the air ejected from the air/water feeding nozzle 19 flowing toward the observation window 15 increase as compared to the endoscope 1E. Accordingly, the endoscope 1F has a further higher cleaning efficiency than the endoscope 1E.

### Second Embodiment

An endoscope 1G of a modification of a second embodiment is similar to the endoscope 1 and the like. Therefore, hereinafter, the components having the same functions as the functions of the components of the endoscope 1 and the like will be assigned the same reference signs and the descriptions will be omitted.

As shown in FIG. 13, in a distal end cover 30G of the endoscope 1G of the present embodiment, the width W31A of the first area 31 where the distance from the air/water feeding nozzle 19 is shortest is greater than the width W31B of the first area 31 where the distance L31 from the air/water feeding nozzle 19 is longest. In the first area 31 and the second area 32, as the distance from the air/water feeding nozzle 19 becomes longer, the width gradually decreases.

In the distal end cover 30G, the center C31 of the first area 31 and the center C32 of the second area 32 are closer to the air/water feeding nozzle 19 as compared to the center C15 of the observation window 15.

As already described, after feeding water, the water is likely to remain around the opening of the air/water feeding nozzle 19. However, the first area 31 with the width W31A has a smaller inclined angle than the first area 31 with the width W31B. Therefore, in the distal end cover 30 of the endoscope 1, after feeding water, the water is unlikely to remain around the opening of the air/water feeding nozzle 19. Thus, in the endoscope 1, the air feeding is efficient.

Note that in the distal end cover 30G of the endoscope 1G, the width W32A of the second area 32 where the distance from the air/water feeding nozzle 19 is shortest is also greater than the width W32B of the second area 32 where the distance L32 from the air/water feeding nozzle 19 is longest.

### Third Embodiment

An endoscope 1H of a third embodiment is similar to and has the same effects as the effects of the endoscope 1 and the like. Therefore, hereinafter, the components having the same functions as the functions of the components of the endoscope 1 and the like will be assigned the same reference signs and the descriptions will be omitted.

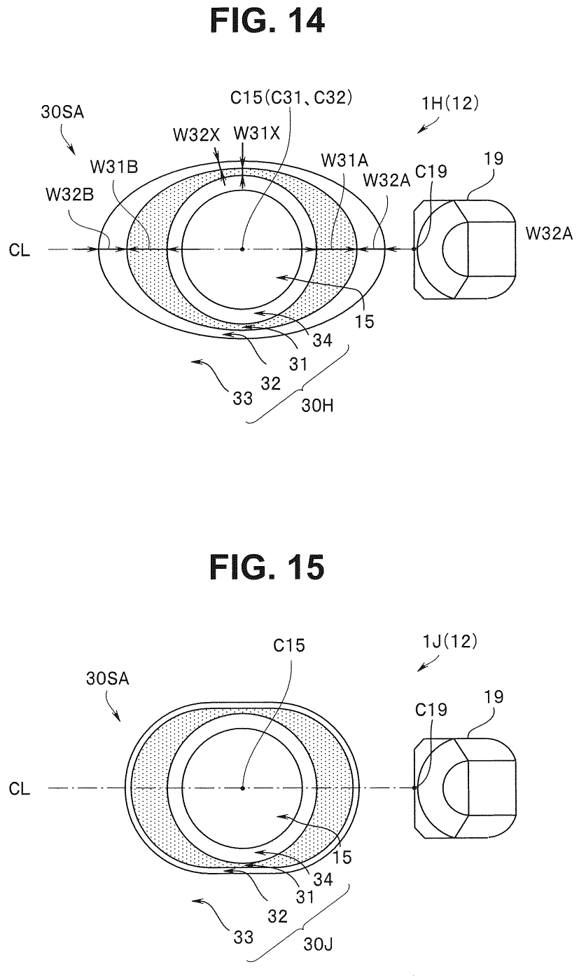

As shown in FIG. 14, in a distal end cover 30H of the endoscope 1H, in the first area 31, the width W31B of a region where the distance from the air/water feeding nozzle 19 is longest and the width W31A of a region where the distance from the air/water feeding nozzle 19 is shortest are both greater than a width W31X of a region between the region with the longest distance and the region with the shortest distance. In the second area 32, the width W32B of a region where the distance from the air/water feeding nozzle 19 is longest and the width W32A of a region where the distance from the air/water feeding nozzle 19 is shortest are both greater than a width W32X of a region between the region with the longest distance and the region with the shortest distance.

In other words, in the distal end cover 30H, in the first area 31, the width of a region closer to the center line CL is wider than the width of a region farther from the center line CL. In the second area 32, the width of a region closer to the center line CL is wider than the width of a region farther from the center line CL.

In the endoscope 1H, after feeding water, the water is unlikely to remain around the opening of the air/water feeding nozzle 19 and the water flowing over the outer surface 15SA of the observation window 15 is also unlikely to remain in the lower portion of the step. Therefore, the air feeding is efficient and even in the endoscope having the optical system with a wide angle of view, water droplets are not seen on an image.

Note that the outer edge of the first area 31 and the outer edge of the second area 32 of the distal end cover 30H are substantially elliptic. However, the shape of the first area 31 and the second area 32 can be modified in various manners.

For example, in an endoscope 1J of a modification of the third embodiment shown in FIG. 15, the outer edge of the first area 31 and the outer edge of the second area 32 of a distal end cover 30J are in such a shape as an athletics track field.

The endoscope 1 of the embodiment may be a flexible endoscope with the insertion portion 6 that is flexible or a rigid endoscope with the insertion portion 6 that is rigid. The endoscope 1 may be for either medical use or industrial use.

The present disclosure is not limited to the aforementioned embodiments and the like, and various changes, combinations, and applications are available within the scope without departing from the gist of the disclosure.

What is claimed is:

1. An endoscope comprising:
an observation window disposed in a distal end portion of an insertion portion;
a nozzle disposed in the distal end portion; and
a cover configured to cover the distal end portion, a distal end surface of the cover includes:
a first surface surrounding the observation window, the first surface having a first planar shape when viewed in a cross-section taken about a line connecting the observation window and the nozzle;
a second surface disposed radially outside the first surface, the second surface having a curved shape when viewed in the cross-section; and
a third surface disposed radially outside of the second surface, the third surface having a second planar shape.

2. The endoscope according to claim 1, wherein the second surface is an inside rounded corner region.

3. The endoscope according to claim 1, wherein the first surface comprising a first width where a distance from the nozzle to an outer periphery of the first surface is longest and a second width where the distance from the nozzle to the outer periphery is shortest, the first width is greater than the second width.

4. The endoscope according to claim 1, wherein a width of the first surface gradually increases as a distance from the nozzle to an outer periphery of the first surface becomes longer.

5. The endoscope according to claim 1, wherein the second surface comprising a first width where a distance from the nozzle to an outer periphery of the second surface is longest and a second width where the distance from the nozzle to the outer periphery is shortest, the first width is greater than the second width.

6. The endoscope according to claim 1, wherein a width of the second surface gradually increases as a distance from the nozzle to an outer periphery of the first surface becomes longer.

7. The endoscope according to claim 1, wherein one or more of a first outer periphery of the first surface and a second outer periphery of the second surface are circular.

8. The endoscope according to claim 1, wherein one or more of a first outer periphery of the first surface and a second outer periphery of the second surface are elliptical.

9. The endoscope according to claim 1, wherein
the distal end surface of the cover further comprises a fourth surface, an inner periphery of the fourth surface contacts the observation window, and an outer periphery of the fourth surface contacts the first surface.

10. The endoscope according to claim 1, wherein a portion of the first surface and a portion of the second surface extend in a direction toward the nozzle.

11. The endoscope according to claim 10, wherein in the portion of the first surface and the portion of the second surface are each equidistant from the nozzle.

12. The endoscope according to claim 1, wherein the first surface comprising a first width where a distance from the nozzle to an outer periphery of the first surface is shortest and a second width where the distance from the nozzle to the outer periphery is longest, the first width is greater than the second width.

13. The endoscope according to claim 12, wherein a width of the first surface gradually decreases as the distance from the nozzle to the outer periphery becomes longer.

14. The endoscope according to claim 12, wherein a width of the second surface gradually decreases as the distance from the nozzle to the outer periphery becomes longer.

15. The endoscope according to claim 1, wherein:
the first surface comprising a first width where a first distance from the nozzle to a first outer periphery of the first surface is longest, and a second width where the first distance is shortest,
the second surface comprising a third width where a second distance from the nozzle to a second outer periphery of the second surface is longest, and a fourth width where the second distance is shortest,
the first surface having a fifth width in a first portion of the first surface between the first width and the second width, wherein the first width and the second width are greater than the fifth width; and
the second surface having a sixth width in a second portion of the second surface between the third width and the fourth width, wherein the third width and the fourth width are greater than the sixth width.

16. The endoscope according to claim 2, wherein the second surface is with a radius of curvature of more than 0.5 mm.

17. The endoscope according to claim 9, wherein the fourth surface has a third planar shape parallel to the second planar shape of the third surface.
